(19) United States
(12) Patent Application Publication
(10) Pub. No.: US 2012/0064517 A1
(43) Pub. Date: Mar. 15, 2012

(54) DETECTION OF CHROMATIN STRUCTURE

(75) Inventor: Steven Okino, San Carlos, CA (US)

(73) Assignee: Bio-Rad Laboratories, Inc., Hercules, CA (US)

(21) Appl. No.: 13/229,312

(22) Filed: Sep. 9, 2011

Related U.S. Application Data
(60) Provisional application No. 61/381,825, filed on Sep. 10, 2010.
(60) Provisional application No. 61/436,138, filed on Jan. 25, 2011.

(51) Int. Cl. C12Q 1/68

(52) U.S. Cl. 435/6.1

(57) ABSTRACT

The present invention provides methods of determining the accessibility of genomic DNA to a DNA modifying agent.

### CROSS-REFERENCE TO RELATED PATENT APPLICATIONS

This application claims benefit of priority to U.S. Provisional Patent Application No. 61/381,825, filed Sep. 10, 2010, and U.S. Provisional Patent Application No. 61/436,138, filed Jan. 25, 2011, each of which is incorporated by reference for all purposes.

### BACKGROUND OF THE INVENTION

Most DNA in a cell is packaged around a set of histone proteins in a coiled structure known as a nucleosome. Nucleosomes, in turn, are further coiled into a highly condensed structure that tightly compacts the DNA. This combination of DNA and protein packaging is generally referred to as chromatin. Chromatin has two forms: euchromatin, a loosely packaged form of chromatin in which the DNA is accessible to transcriptional machinery and is usually, but not always, transcriptionally active, and heterochromatin, a tightly packaged form in which the DNA is inaccessible to transcriptional machinery and is usually, but not always, transcriptionally silent.

The transition between euchromatin and heterochromatin is mainly controlled by three epigenetic events, DNA methylation, histone modification, and RNA interaction. These epigenetic events affect whether genomic DNA in a cell is in a loosely packaged, transcriptionally active form or a tightly packaged, transcriptionally silent form.

### BRIEF SUMMARY OF THE INVENTION

The present invention provides methods for analyzing chromosomal DNA. In some embodiments, the method comprises:
(a) introducing a DNA modifying agent into a nucleus having genomic DNA under conditions such that the DNA modifying agent modifies the genomic DNA in the nucleus, wherein different regions of the genomic DNA are modified to a different extent by the DNA modifying agent, thereby forming modified DNA; and
(b) nucleotide sequencing at least one DNA region in the modified DNA, wherein the sequencing comprises simultaneously determining (1) the nucleotide sequence and (2) whether sequenced nucleotides are modified.

In some embodiments, the nucleus is an isolated nucleus. In some embodiments, the nucleus is in a cell.

In some embodiments, before or during step (a), the method comprises permeabilizing or disrupting a cell membrane of the cell, and step (a) comprises contacting the cell with the DNA modifying agent. In some embodiments, step (a) comprises expressing the DNA modifying agent in the cell, thereby introducing the DNA modifying agent into the cell.

In some embodiments, the modifying agent is a DNA methyltransferase. In some embodiments, the DNA methyltransferase methylates adenosines in DNA. In some embodiments, the DNA methyltransferase methylates cytosines in DNA.

In some embodiments, the sequencing comprises monitoring DNA polymerase kinetics. In some embodiments, the sequencing does not utilize a polymerase.

In some embodiments, the sequencing comprises nanopore sequencing.

In some embodiments, the sequencing comprises template-dependent replication of the DNA region that results in incorporation of labeled nucleotides, and wherein an arrival time and/or duration of an interval between signal generated from different incorporated nucleotides is determinative of the presence or absence of the modification and/or the identity of an incorporated nucleotide. In some embodiments, the label of the labeled nucleotides is a fluorescent label.

In some embodiments, the permeabilizing step comprises contacting the cell with an agent that permeabilizes the cell membrane. In some embodiments, the agent that permeabilizes the cell membrane is a lysolipid. In some embodiments, the permeabilizing or disrupting and the contacting of the cell with a DNA modifying agent are performed simultaneously.

In some embodiments, the method further comprises quantifying the extent of modification in at least one DNA region as compared to a control DNA region, wherein the control DNA region comprises a sequence that is either:
(i) accessible in essentially all cells of an animal; or
(ii) inaccessible in essentially all cells of an animal.

The present invention also provides a method of analyzing chromosomal DNA in a cell, comprising:
(a) introducing a DNA modifying agent into a nucleus having genomic DNA under conditions such that the DNA modifying agent modifies the genomic DNA in the nucleus, wherein different regions of the genomic DNA are modified to a different extent by the DNA modifying agent, thereby forming modified DNA;
(b) purifying the DNA thereby generating purified DNA;
(c) fragmenting the purified DNA;
(d) affinity purifying modified DNA from the purified and fragmented DNA, thereby generating a DNA sample enriched for modified DNA; and
(e) detecting a presence, absence, or quantity of one or more DNA region in the DNA sample enriched for modified DNA or cloning, isolating, or nucleotide sequencing at least one DNA fragment from the DNA sample enriched for modified DNA.

In some embodiments, the nucleus is an isolated nucleus. In some embodiments, the nucleus is in a cell. In some embodiments, before or during step (a), the method comprises permeabilizing or disrupting a cell membrane of the cell, and wherein step (a) comprises contacting the cell with the DNA modifying agent. In some embodiments, step (a) comprises expressing the DNA modifying agent in the cell, thereby introducing the DNA modifying agent into the cell.

In some embodiments, the modifying agent is a DNA methyltransferase. In some embodiments, the DNA methyltransferase methylates adenosines in DNA. In some embodiments, the DNA methyltransferase methylates cytosines in DNA.

In some embodiments, the affinity purifying comprises contacting the fragmented and purified DNA with a protein affinity agent having affinity for modified DNA under conditions to allow for binding of the affinity agent to modified DNA, and removing DNA that does not bind to the affinity agent. In some embodiments, the protein affinity agent comprises an antibody specific for modified DNA. In some embodiments, the modification of the modified DNA is methylation of adenosine or methylation of cytosine.

In some embodiments, the detecting step comprises detecting the quantity of copies of at least one DNA region in the DNA sample enriched for modified DNA.

In some embodiments, the method comprises amplifying the at least one DNA region. In some embodiments, the amplifying step comprises real-time PCR.

In some embodiments, the detecting step comprises nucleotide sequencing at least one DNA region. In some embodiments, the nucleotide sequencing comprises monitoring DNA polymerase kinetics. In some embodiments, the nucleotide sequencing comprises simultaneously determining (1) the nucleotide sequence and (2) whether sequenced nucleotides are modified.

In some embodiments, the detecting step comprises hybridizing the DNA sample enriched for modified DNA to a plurality of nucleic acid probes and detecting hybridization between the DNA sample and the nucleic acid probes. In some embodiments, the nucleic acid probes are linked to a solid support. In some embodiments, the solid support is selected from the group consisting of a microarray and beads.

In some embodiments, the fragmenting comprises shearing or sonicating the DNA or digesting the DNA with a sequence non-specific nuclease.

### DEFINITIONS

A “DNA modifying agent,” as used herein, refers to a molecule that alters DNA in a detectable manner. In some embodiments, the DNA modifying agent is a molecule that methylates specific bases within a DNA strand at specific positions. Exemplary DNA modifying agents include, but are not limited to, enzymes, proteins, and chemicals.

A “DNA region,” as used herein, refers to a target sequence of interest within genomic DNA. The DNA region can be of any length that is of interest and that is accessible by the DNA modifying agent being used. In some embodiments, the DNA region can include a single base pair, but can also be a short segment of sequence within genomic DNA (e.g., 2-100, 2-500, 50-500 bp) or a larger segment (e.g., 100-10,000, 100-1000, or 1000-5000 bp). The amount of DNA in a DNA region is sometimes determined by the amount of sequence to be amplified in a PCR reaction. For example, standard PCR reactions generally can amplify between about 35 to 5000 base pairs.

A different “extent” of modifications refers to a different number (actual or relative) of modified copies of one or more DNA regions between samples or between two or more DNA regions in one or more samples. For example, if 100 copies of two DNA regions (designated for convenience as “region A” and “region B”) are each present in chromosomal DNA in a cell, an example of modification to a different extent would be if 10 copies of region A were modified whereas 70 copies of region B were modified.

“Permeabilizing” a membrane, as used herein, refers to reducing the integrity of a cell membrane to allow for entry of a modifying agent into the cell. A cell with a permeabilized cell membrane will generally retain the cell membrane such that the cell's structure remains substantially intact. In contrast, “disrupting” a cell membrane, as used herein, refers to reducing the integrity of a cell membrane such that the cell's structure does not remain intact. For example, contacting a cell membrane with a nonionic detergent will remove and/or dissolve a cell membrane, thereby allowing access of a modifying agent to genomic DNA that retains at least some chromosomal structure.

The terms “oligonucleotide,” “polynucleotide,” and “nucleic acid” interchangeably refer to a polymer of monomers that can be corresponded to a ribose nucleic acid (RNA) or deoxyribose nucleic acid (DNA) polymer, or analog thereof. This includes polymers of nucleotides such as RNA and DNA, as well as modified forms thereof, peptide nucleic acids (PNAs), locked nucleic acids (LNA™), and the like. In certain applications, the nucleic acid can be a polymer that includes multiple monomer types, e.g., both RNA and DNA subunits.

A nucleic acid is typically single-stranded or double-stranded and will generally contain phosphodiester bonds, although in some cases, as outlined herein, nucleic acid analogs are included that may have alternate backbones, including, for example and without limitation, phosphoramide (Beaucage et al. (1993) Tetrahedron 49(10):1925 and the references therein; Letsinger (1970) J. Org. Chem. 35:3800; Sprinzl et al. (1977) Eur. J. Biochem. 81:579; Letsinger et al. (1986) Nucl. Acids Res. 14: 3487; Sawai et al. (1984) Chem. Lett. 805; Letsinger et al. (1988) J. Am. Chem. Soc. 110:4470; and Pauwels et al. (1986) Chemica Scripta 26:1419), phosphorothioate (Mag et al. (1991) Nucleic Acids Res. 19:1437 and U.S. Pat. No. 5,644,048), phosphorodithioate (Briu et al. (1989) J. Am. Chem. Soc. 111:2321), O-methylphophoroamidite linkages (Eckstein, Oligonucleotides and Analogues: A Practical Approach, Oxford University Press (1992)), and peptide nucleic acid backbones and linkages (Egholm (1992) J. Am. Chem. Soc. 114:1895; Meier et al. (1992) Chem. Int. Ed. Engl. 31:1008; Nielsen (1993) Nature 365:566; and Carlsson et al. (1996) Nature 380:207), which references are each incorporated by reference. Other analog nucleic acids include those with positively charged backbones (Denpcy et al. (1995) Proc. Natl. Acad. Sci. USA 92:6097); non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghvi and P. Dan Cook; Mesmaeker et al. (1994) Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghvi and P. Dan Cook, which references are each incorporated by reference. Nucleic acids containing one or more carbocyclic sugars are also included within the definition of nucleic acids (Jenkins et al. (1995) Chem. Soc. Rev. pp169-176, which is incorporated by reference). Several nucleic acid analogs are also described in, e.g., Rawls, C & E News Jun. 2, 1997 page 35, which is incorporated by reference. These modifications of the ribose-phosphate backbone may be done to facilitate the addition of additional moieties such as labeling moieties, or to alter the stability and half-life of such molecules in physiological environments.

In addition to naturally occurring heterocyclic bases that are typically found in nucleic acids (e.g., adenine, guanine, thymine, cytosine, and uracil), nucleic acid analogs also include those having non-naturally occurring heterocyclic or other modified bases, many of which are described, or otherwise referred to, herein. In particular, many non-naturally occurring bases are described further in, e.g., Seela et al. (1991) Helv. Chim. Acta 74:1790, Grein et al. (1994) Bioorg. Med. Chem. Lett. 4:971-976, and Seela et al. (1999) Helv. Chim. Acta 82:1640, which are each incorporated by reference. To further illustrate, certain bases used in nucleotides that act as melting temperature (Tm) modifiers are optionally included. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, entitled “SYNTHESIS OF 7-DEAZA-2′-DEOXYGUANOSINE NUCLEOTIDES,” which issued Nov. 23, 1999 to Seela, which is incorporated by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytosine; 5-fluorocytosine; 5-chlorocytosine; 5-iodocytosine; 5-bromocytosine; 5-methylcytosine; 5-propynylcytosine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

“Accessibility” of a DNA region to a DNA modifying agent, as used herein, refers to the ability of a particular DNA region in a chromosome of a cell to be contacted and modified by a particular DNA modifying agent. Without intending to limit the scope of the invention, it is believed that the particular chromatin structure comprising the DNA region will affect the ability of a DNA modifying agent to modify the particular DNA region. For example, the DNA region may be wrapped around histone proteins and further may have additional nucleosomal structure that prevents, or reduces access of, the DNA modifying agent to the DNA region of interest.

“Nucleotide sequencing,” as used herein, refers to a process of determining the nucleotide composition of a polynucleotide or nucleic acid fragment. In some embodiments, nucleotide sequencing comprises determining both the order of nucleotides of a particular nucleic acid fragment and whether one or more of the sequenced nucleotides are modified, e.g., by methylation of a nucleotide at a specific position. Exemplary nucleotide sequencing methods of the present invention include, but are not limited to, single-molecule real-time sequencing and nanopore sequencing.

“DNA polymerase kinetics,” as used herein, refers to the rate of DNA synthesis by a DNA polymerase. The rate of DNA synthesis is influenced by numerous factors, including nucleotide binding and polymerase translocation, as well as by the presence of modified nucleotides (e.g., methylated nucleotides), which decrease the rate of DNA synthesis. “Monitoring DNA polymerase kinetics,” as used herein, refers to a method of measuring the rate of DNA synthesis by a DNA polymerase. In some embodiments, the rate of DNA synthesis is monitored in real time. In some embodiments, DNA polymerase kinetics are measured by fluorescently labeling nucleotides and measuring the fluorescence pulse of a nucleotide as it is incorporated into the growing DNA strand. DNA polymerase kinetics can be measured by any of several metrics, including but not limited to pulse width (the duration of a fluorescence pulse) and interpulse duration (the interval between successive pulses).

### BRIEF DESCRIPTION OF THE DRAWINGS

FIG. 1. Principle of DNA modification. (A) Cells are treated with a DNA modifying agent in situ to modify accessible chromatin; inaccessible chromatin regions are refractory to modification. (B) The modified DNA is purified and sequenced directly using technology that can detect sites of DNA modification.

FIG. 2A-D. DAM modification of accessible chromatin. Permeabilized cells were treated in situ with the DAM methyltransferase to modify accessible chromatin (DAM methylates the A residue at the 6-position in a GATC motif); control cells were treated with permeabilization buffer only. The DNA was purified and digested with DpnII, a methylation-sensitive restriction enzyme that only digests GATC motifs that have not been DAM modified; control reactions were treated with buffer only. The DNA samples were then amplified using primers specific for the B2M (A), RHO (B), p14 (C), and CDH13 (D) promoters. Triangle, no DAM/no DpnII; square, no DAM/plus DpnII; diamond, plus DAM/no DpnII; circle, plus DAM/plus DpnII.

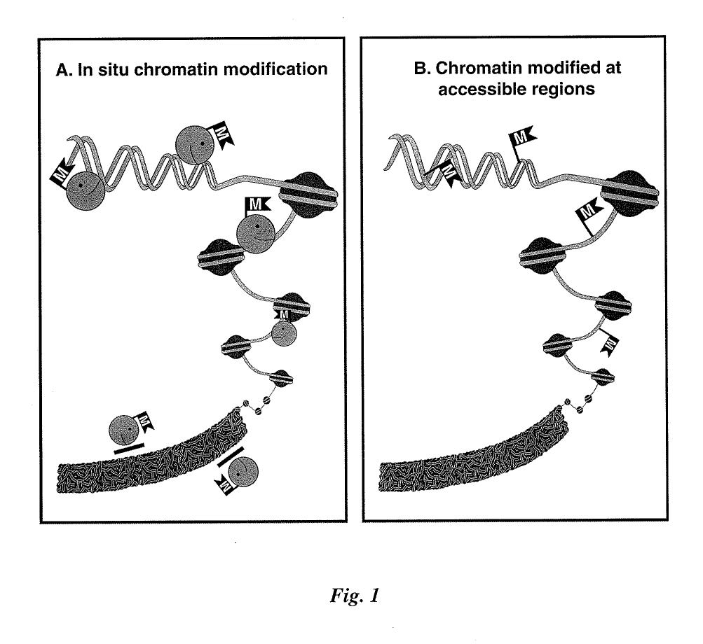

### DETAILED DESCRIPTION

### I. Introduction

Methods are provided for analyzing chromatin structure of chromosomal DNA by modifying genomic DNA in a nucleus with a DNA modifying agent and then nucleotide sequencing at least one DNA region in the modified DNA. The extent of modification in a DNA region can be quantified and is indicative of the accessibility of that region of DNA to the modifying agent, and thus reflects the chromatin structure of that region.

One advantage of the present invention is that one can analyze modified DNA and simultaneously determine the nucleotide sequence of the DNA strand and whether the sequenced nucleotides are modified, e.g., methylated. This direct detection of modified bases during sequencing allows for the rapid generation of results.

### II. General Method

The methods of the invention can involve introducing a DNA modifying agent into a nucleus having genomic DNA under conditions such that the DNA modifying agent modifies the genomic DNA in the nucleus, wherein different regions of the genomic DNA are modified to a different extent by the DNA modifying agent (due to differences in chromatin structure) and then nucleotide sequencing at least one DNA region in the modified DNA, wherein the sequencing comprises simultaneously determining the nucleotide sequence and whether sequenced nucleotides are modified. In some embodiments, the extent of modification in at least one DNA region is quantified. The varying accessibility of the DNA can reflect the nucleosomal/chromosomal structure of the genomic DNA. For example, in some embodiments, DNA regions that are more accessible to DNA modifying agents are likely in more “loose” chromatin structures.

In some embodiments, the nucleotide sequencing step comprises monitoring DNA polymerase kinetics. DNA polymerization for a DNA sequence of interest can be observed in real-time, e.g., using a single-molecule, real-time (SMRT) sequencing method. In some embodiments, nucleotide-specific differences in catalyzing the incorporation of nucleotides can be detected and correlated with the identity of an incorporated nucleotide and/or the presence or absence of a modification of an incorporated nucleotide.

In some embodiments, the nucleotide sequencing step comprises nanopore sequencing. A DNA sequence of interest can be threaded through a nanopore, e.g., a protein nanopore, under an applied potential while recording modulations of the ionic current passing through the pore. Because modulations in pore current and dwell time differ for varying nucleotides, these modulations in pore current and dwell times can be detected and correlated with the identity of an incorporated nucleotide and/or the presence or absence of a modification of an incorporated nucleotide.

In some embodiments, the nucleus into which the DNA modifying agent is introduced is an isolated nucleus. In some embodiments, the nucleus is in a cell.

When the nucleus into which the DNA modifying agent is introduced is in a cell, the methods of the invention can include permeabilizing or disrupting a cell membrane of the cell, thereby introducing the agent into the cell and/or enhancing introduction of the agent into the cell. The permeabilization or disruption of the cell membrane can occur before the DNA modifying agent is introduced into the cell, or permeabilization or disruption of the cell membrane can occur simultaneously with the introduction of the DNA modifying agent into the cell. Alternatively, the DNA modifying agent can be introduced into isolated nuclei.

A variety of eukaryotic cells can be used in the present invention. In some embodiments, the cells are animal cells, including but not limited to, human, or non-human, mammalian cells. Non-human mammalian cells include but are not limited to, primate cells, mouse cells, rat cells, porcine cells, and bovine cells. In some embodiments, the cells are plant or fungal (including but not limited to yeast) cells. Cells can be, for example, cultured primary cells, immortalized culture cells or can be from a biopsy or tissue sample, optionally cultured and stimulated to divide before assayed. Cultured cells can be in suspension or adherent prior to and/or during the permeabilization and/or DNA modification steps. Cells can be from animal tissues, biopsies, etc. For example, the cells can be from a tumor biopsy.

The present invention also provides for a method of analyzing chromosomal DNA in a cell by (1) introducing a DNA modifying agent into the nucleus of the cell such that the DNA modifying agent modifies some genomic regions more than others (e.g., due to steric hindrance due to variations in chromatin structure), (2) affinity purifying the modified DNA using an affinity agent specific for the DNA modification, and then subsequently (3) analyzing the affinity purified sample enriched for modified DNA.

As explained herein, a DNA modifying agent can be introduced into a cell's nucleus by a number of methods. Once the genomic DNA in the nucleus has been modified, the DNA can be purified, e.g., through standard molecular biology methods, and optionally fragmented. Fragmentation can be achieved, for example, by DNA shearing (e.g., extruding the DNA through a small-gauge needle), sonication, or cleavage with a nucleic acid nuclease (e.g., a DNase).

Once purified, and optionally fragmented, the DNA can be submitted to one or more affinity purification steps using an affinity agent specific for the DNA modification. DNA fragments containing one or more DNA modification will thereby become enriched in the sample, while fragments having few or no modifications can be washed away. The DNA in the resulting enriched sample can subsequently be analyzed. Sequences enriched in the enriched sample will likely have a more “open” chromatin conformation in the cell from which the DNA was obtained such that the DNA modifying agent could contact and modify the sequence.

DNA affinity agents can be any molecule that has a selective affinity for the DNA modification. In some embodiments, the affinity agent is an antibody. For example, antibodies having affinity for methyl-cytosine and methyl-adenosine are known and commercially available. Antibodies specific for other types of DNA modifications are also contemplated. Alternatively, the affinity agent can be a non-antibody protein. As an example, methyl binding protein (MBP) can be used where the DNA modification is methyl-cytosine. In yet other embodiments, the affinity agent is a non-protein molecule, such as a carbohydrate, lipid, nucleic acid (including but not limited to an aptamer) or other molecule.

In some embodiments, the affinity agent is linked to a solid support. In some embodiments, the fragmented and purified DNA is contacted to the affinity agent linked to the solid support under conditions to allow the affinity agent to bind to modified DNA, and unbound DNA is washed or otherwise separated from the bound DNA.

### III. DNA Modifying Agents

According to the methods of the present invention, a DNA modifying agent is introduced into a nucleus having genomic DNA under such conditions that the DNA modifying agent modifies the genomic DNA in the nucleus. A wide variety of DNA modifying agents can be used according to the present invention, including but not limited to enzymes, proteins, and chemicals.

In some embodiments, the DNA modifying agent is introduced into an isolated nucleus. In some embodiments, the DNA modifying agent is introduced into a nucleus in a cell following permeabilization, or simultaneously with permeabilization (e.g., during electroporation or during incubation with permeabilizing agent).

In some embodiments, the DNA modifying agents are contacted to permeabilized cells following removal of the permeabilizing agent, optionally with a change of the buffer. Alternatively, in some preferred embodiments, the DNA modifying agent is contacted to the genomic DNA without one or more intervening steps (e.g., without an exchange of buffers, washing of the cells, etc.). As noted above, this latter approach can be convenient for reducing the amount of labor and time necessary and also removes a potential source of error and contamination in the assay.

The quantity of DNA modifying agent used, as well as the time of the reaction with the DNA modifying agent will depend on the agent used. Those of skill in the art will appreciate how to adjust conditions depending on the agent used. Generally, the conditions of the DNA modifying step are adjusted such that a “complete” modification is not achieved. Thus, for example, in some embodiments, the conditions of the modifying step is set such that for the positive control—i.e., the control where modification is accessible and occurs—the number of copies of that positive control DNA region that are modified is at least about 10%, at least about 15%, 20%, 25%, 30%, 40%, or more.

### A. Methyltransferases

In some embodiments of the invention, the DNA modifying agent generates a covalent modification to the DNA. For example, in some embodiments, the DNA modifying agents of the invention are methyltransferases. A variety of methyltransferases are known in the art and can be used in the invention.

In some embodiments, the methyltransferase used adds a methyl moiety to adenosine in DNA. Examples of such methyltransferases include, but are not limited to, E. coli DAM methyltransferase, M.TaqI, M.EcoRV, M.FokI, and M.EcoRI. Because adenosine generally is not methylated in eukaryotic cells, the presence of a methylated adenosine in a particular DNA region indicates that a DAM methyltransferase, M.TaqI, M.EcoRV, M.FokI, and M.EcoRI (or other methyltransferase with similar activity) was able to access the DNA region.

In some embodiments, the methyltransferase methylates cytosines in GC sequences. Examples of such methyltransferases include, but are not limited to, M.CviPI. See, e.g., Xu et al., Nuc. Acids Res. 26(17): 3961-3966 (1998). Because GC sequences generally are not methylated in eukaryotic cells, the presence of a methylated GC sequence in a particular DNA region indicates that the DNA modifying agent (i.e., a methyltransferase that methylates cytosines in GC sequences) was able to access the DNA region.

In some embodiments, the methyltransferase methylates cytosines in CG (also known as “CpG”) sequences. Examples of such methyltransferases include, but are not limited to, M.SssI. Use of such methyltransferases will generally be limited to use for those DNA regions that are not typically methylated. This is because CG sequences are endogenously methylated in eukaryotic cells and thus it is not generally possible to assume that a CG sequence is methylated by the modifying agent rather than an endogenous methyltransferase except in such DNA regions where methylation is rare.

Other suitable methyltransferases that are known in the art include, for example, methyltransferases that methylate cytosine at the N4 position (e.g., M.BamHI and M.PvuII) and methyltransferases that methylate cytosine at the C5 position (e.g., M.HhaI). Alternatively, mutated or genetically engineered methyltransferases that exhibit altered DNA target-site specificity or altered DNA modification specificity can be used.

### B. Chemicals

In some embodiments, the DNA modifying agent comprises a DNA modifying chemical. As most DNA modifying chemicals are relatively small compared to chromatin, use of DNA modifying chemicals without a fusion partner may not be effective in some circumstances as there will be little if any difference in the extent of accessibility of different DNA regions. Therefore, in some embodiments, the DNA modifying agent comprises a molecule having steric hindrance linked to a DNA modifying chemical. The molecule having steric hindrance can be any protein or other molecule that results in differential accessibility of the DNA modifying agent depending on chromatin structure. This can be tested, for example, by comparing results to those using a methyltransferase as described herein.

In some embodiments, the molecule having steric hindrance will be at least 5, 7, 10, or 15 kD in size. Those of skill in the art will likely find it convenient to use a polypeptide as the molecule with steric hindrance. Any polypeptide can be used that does not significantly interfere with the DNA modifying agent's ability to modify DNA. In some embodiments, the polypeptide is a double-stranded sequence-non-specific nucleic acid binding domain as discussed in further detail below.

The DNA modifying chemicals of the present invention can be linked directly to the molecule having steric hindrance or via a linker. A variety of homo- and hetero-bifunctional linkers are known and can be used for this purpose.

Exemplary DNA modifying chemicals include but are not limited to hydrazine (and derivatives thereof, e.g., as described in Mathison et al., Toxicology and Applied Pharmacology 127(1):91-98 (1994)) and dimethyl sulfate. In some embodiments, hydrazine introduces a methyl group to guanine in DNA or otherwise damages DNA. In some embodiments, dimethyl sulfate methylates guanine or results in the base-specific cleavage of guanine in DNA by rupturing the imidazole rings present in guanine.

### C. DNA Binding Domains to Improve DNA Modifying Agents

In some embodiments, the DNA modifying agents of the invention are fused or otherwise linked to a double-stranded sequence-non-specific nucleic acid binding domain (e.g., a DNA binding domain). In cases where the DNA modifying agent is a polypeptide, the double-stranded sequence-non-specific nucleic acid binding domain can be synthesized, for example, as a protein fusion with the DNA modifying agent via recombinant DNA technology. A double-stranded sequence-non-specific nucleic acid binding domain is a protein or defined region of a protein that binds to double-stranded nucleic acid in a sequence-independent manner, i.e., binding does not exhibit a gross preference for a particular sequence. In some embodiments, double-stranded nucleic acid binding proteins exhibit a 10-fold or higher affinity for double-stranded versus single-stranded nucleic acids. The double-stranded nucleic acid binding proteins in some embodiments of the invention are thermostable. Examples of such proteins include, but are not limited to, the Archaeal small basic DNA binding proteins Sac7d and Sso7d (see, e.g., Choli et al., Biochimica et Biophysica Acta 950:193-203, 1988; Baumann et al., Structural Biol. 1:808-819, 1994; and Gao et al, Nature Struc. Biol. 5:782-786, 1998), Archael HMf-like proteins (see, e.g., Starich et al., J. Molec. Biol. 255:187-203, 1996; Sandman et al., Gene 150:207-208, 1994), and PCNA homologs (see, e.g., Cann et al., J. Bacteriology 181:6591-6599, 1999; Shamoo and Steitz, Cell:99, 155-166, 1999; De Felice et al., J. Molec. Biol. 291, 47-57, 1999; and Zhang et al., Biochemistry 34:10703-10712, 1995). See also European Patent 1283875B1 for addition information regarding DNA binding domains.

### Sso7d and Sac7d

Sso7d and Sac7d are small (about 7,000 kd MW), basic chromosomal proteins from the hyperthermophilic archaeabacteria Sulfolobus solfataricus and S. acidocaldarius, respectively. These proteins are lysine-rich and have high thermal, acid and chemical stability. They bind DNA in a sequence-independent manner and when bound, increase the T_{M} of DNA by up to 40° C. under some conditions (McAfee et al., Biochemistry 34:10063-10077, 1995). These proteins and their homologs are typically believed to be involved in stabilizing genomic DNA at elevated temperatures.

### HMf-Like Proteins

The HMf-like proteins are archaeal histones that share homology both in amino acid sequences and in structure with eukaryotic H4 histones, which are thought to interact directly with DNA. The HMf family of proteins form stable dimers in solution, and several HMf homologs have been identified from thermostable species (e.g., Methanothermus fervidus and Pyrococcus strain GB-3a). The HMf family of proteins, once joined to Taq DNA polymerase or any DNA modifying enzyme with a low intrinsic processivity, can enhance the ability of the enzyme to slide along the DNA substrate and thus increase its processivity. For example, the dimeric HMf-like protein can be covalently linked to the N terminus of Taq DNA polymerase, e.g., via chemical modification, and thus improve the processivity of the polymerase.

Those of skill in the art will recognize that other double-stranded sequence-non-specific nucleic acid binding domains are known in the art and can also be used as described herein.

### IV. Permeabilizing and Disrupting Cells

Cell membranes can be permeabilized or disrupted in any way known in the art. As explained herein, the present methods involve contacting the genomic DNA prior to isolation of the DNA and thus methods of permeabilizing or disrupting the cell membrane will not disrupt the structure of the genomic DNA of the cell such that nucleosomal or chromatin structure is destroyed.

In some embodiments, the cell membrane is contacted with an agent that permeabilizes or disrupts the cell membrane. Lysolipids are an exemplary class of agents that permeabilize cell membranes. Exemplary lysolipids include, but are not limited to, lysophosphatidylcholine (also known in the art as lysolecithin) or monopalmitoylphosphatidylcholine. A variety of lysolipids are also described in, e.g., WO/2003/052095.

Non-ionic detergents are an exemplary class of agents that disrupt cell membranes. Exemplary non-ionic detergents, include but are not limited to, NP40, Tween 20 and Triton X-100.

In some embodiments, the permeabilization agent and the DNA modifying agent are delivered simultaneously. Thus, in some embodiments, a buffer comprising both agents is contacted to the cell. The buffer should be adapted for maintaining activity of both agents while maintaining the structure of the cellular chromatin.

Alternatively, electroporation or biolistic methods can be used to permeabilize a cell membrane such that a DNA modifying agent is introduced into the cell and can thus contact the genomic DNA. A wide variety of electroporation methods are well known and can be adapted for delivery of DNA modifying agents as described herein. Exemplary electroporation methods include, but are not limited to, those described in WO/2000/062855. Biolistic methods include but are not limited to those described in U.S. Pat. No. 5,179,022.

### V. Analyzing DNA after DNA Modification Step

In some embodiments, following the DNA modification step, genomic DNA is isolated from the nucleus according to any method available. Essentially any DNA purification procedure can be used so long as it results in DNA of acceptable purity for the subsequent sequencing step. For example, standard cell lysis reagents can be used to lyse cells. Optionally a protease (including but not limited to proteinase K) can be used. DNA can be isolated from the mixture as is known in the art. In some embodiments, phenol/chloroform extractions are used and the DNA can be subsequently precipitated (e.g., by ethanol) and purified. In some embodiments, RNA is removed or degraded (e.g., with an RNase or with use of a DNA purification column), if desired.

### A. Target DNA Region

In some embodiments, the methods of the present invention are utilized to sequence the whole genome. Alternatively, in some embodiments, the methods of the present invention are utilized to sequence a target DNA region. A DNA region is a target sequence of interest within genomic DNA. Any DNA sequence in genomic DNA can be evaluated for DNA modifying agent accessibility as described herein. DNA regions can be screened to identify a DNA region of interest that displays different accessibility in different cell types, between untreated cells and cells exposed to a drug, chemical or environmental stimulus, or between normal and diseased tissue, for example. Thus, in some embodiments, the methods of the invention are used to identify a DNA region whose change in accessibility acts as a marker for disease (or lack thereof). Exemplary diseases include but are not limited to cancers. A number of genes have been described that have altered DNA methylation and/or chromatin structure in cancer cells compared to non-cancer cells.

A variety of DNA regions can be detected either for research purposes and/or as a control DNA region to confirm that the reagents were performing as expected. For example, in some embodiments, a DNA region is assayed that is accessible in essentially all cells of an animal. Such DNA regions are useful, for example, as positive controls for accessibility. Such DNA regions can be found, for example, within or adjacent to genes that are constitutive or nearly constitutive. Such genes include those generally referred to as “housekeeping” genes, i.e., genes whose expression are required to maintain basic cellular function. Examples of such genes include, but are not limited to, glyceraldehyde-3-phosphate dehydrogenase (GAPDH) and beta actin (ACTB). DNA regions can include all or a portion of such genes, optionally including at least a portion of the promoter.

In some embodiments, a DNA region comprises at least a portion of DNA that is inaccessible in most cells of an animal. Such DNA regions are useful, for example, as negative controls for accessibility. “Inaccessible” in this context refers to DNA regions whose copies are modified in no more than around 5% of the copies of the DNA region. Examples of such gene sequences include those generally recognized as “heterochromatic” and include genes that are only expressed in very specific cell types (e.g., expressed in a tissue or organ-specific fashion). Exemplary genes that are generally inaccessible (with the exception of specific cell types) include, but are not limited to, hemoglobin-beta chain (HBB), immunoglobulin light chain kappa (IGK), and rhodopsin (RHO).

In some embodiments, the DNA region is a gene sequence which has different accessibility depending on the disease state of the cell or otherwise have variable accessibility depending on type of cells or growth environment. For example, some genes are generally inaccessible in non-cancer cells but are accessible in cancer cells. Examples of genes with variable accessibility include, e.g., glutathione-s-transferase pi (GSTP1).

In some embodiments, the DNA regions are selected at random, for example, to identify regions that have differential accessibility between different cell types, different conditions, normal vs. diseased cells, etc.

### B. Nucleotide Sequencing

A variety of methods can be used to determine the nucleotide sequence and the extent to which sequenced nucleotides are modified, e.g., methylated. Any sequencing method known in the art can be used so long as it can simultaneously determine the nucleotide sequence and whether sequenced nucleotides are modified. As used herein, “simultaneously” means that as the sequencing process determines the order of nucleotides in a nucleic acid fragment, at the same time it can also distinguish between modified nucleotides (e.g., methylated nucleotides) and non-modified nucleotides (e.g., non-methylated nucleotides). Examples of sequencing processes that can simultaneous detect nucleotide sequence and distinguish whether sequenced nucleotides are modified include, but are not limited to, single-molecule real-time (SMRT) sequencing and nanopore sequencing.

In some embodiments, nucleotide sequencing comprises template-dependent replication of the DNA region that results in incorporation of labeled nucleotides (e.g., fluorescently labeled nucleotides), and wherein an arrival time and/or duration of an interval between signal generated from different incorporated nucleotides is determinative of the presence or absence of the modification and/or the identity of an incorporated nucleotide.

### Single-Molecule, Real-Time Sequencing

In some embodiments, genomic DNA comprising a target DNA region is sequenced by single-molecule, real-time (SMRT) sequencing. SMRT sequencing is a process by which single DNA polymerase molecules are observed in real time while they catalyze the incorporation of fluorescently labeled nucleotides complementary to a template nucleic acid strand. Methods of SMRT sequencing are known in the art and were initially described by Flusberg et al., Nature Methods, 7:461-465 (2010), which is incorporated herein by reference for all purposes.

Briefly, in SMRT sequencing, incorporation of a nucleotide is detected as a pulse of fluorescence whose color identifies that nucleotide. The pulse ends when the fluorophore, which is linked to the nucleotide's terminal phosphate, is cleaved by the polymerase before the polymerase translocates to the next base in the DNA template. Fluorescence pulses are characterized by emission spectra as well as by the duration of the pulse (“pulse width”) and the interval between successive pulses (“interpulse duration” or “IPD”). Pulse width is a function of all kinetic steps after nucleotide binding and up to fluorophore release, and IPD is a function of the kinetics of nucleotide binding and polymerase translocation. Thus, DNA polymerase kinetics can be monitored by measuring the fluorescence pulses in SMRT sequencing.

In addition to measuring differences in fluorescence pulse characteristics for each fluorescently-labeled nucleotide (i.e., adenine, guanine, thymine, and cytosine), differences can also be measured for non-methylated versus methylated bases. For example, the presence of a methylated base alters the IPD of the methylated base as compared to its non-methylated counterpart (e.g., methylated adenosine as compared to non-methylated adenosine). Additionally, the presence of a methylated base alters the pulse width of the methylated base as compared to its non-methylated counterpart (e.g., methylated cytosine as compared to non-methylated cytosine) and furthermore, different modifications have different pulse widths (e.g., 5-hydroxymethylcytosine has a more pronounced excursion than 5-methylcytosine). Thus, each type of non-modified base and modified base has a unique signature based on its combination of IPD and pulse width in a given context. The sensitivity of SMRT sequencing can be further enhanced by optimizing solution conditions, polymerase mutations and algorithmic approaches that take advantage of the nucleotides' kinetic signatures, and deconvolution techniques to help resolve neighboring methylcytosine bases.

### Nanopore Sequencing

In some embodiments, nucleotide sequencing does not comprise template-dependent replication of a DNA region. In some embodiments, genomic DNA comprising a target DNA region is sequenced by nanopore sequencing. Nanopore sequencing is a process by which a polynucleotide or nucleic acid fragment is passed through a pore (such as a protein pore) under an applied potential while recording modulations of the ionic current passing through the pore. Methods of nanopore sequencing are known in the art; see, e.g., Clarke et al., Nature Nanotechnology 4:265-270 (2009), which is incorporated herein by reference for all purposes.

Briefly, in nanopore sequencing, as a single-stranded DNA molecule passes through a protein pore, each base is registered, in sequence, by a characteristic decrease in current amplitude which results from the extent to which each base blocks the pore. An individual nucleobase can be identified on a static strand, and by sufficiently slowing the rate of speed of the DNA translocation (e.g., through the use of enzymes) or improving the rate of DNA capture by the pore (e.g., by mutating key residues within the protein pore), an individual nucleobase can also be identified while moving.

In some embodiments, nanopore sequencing comprises the use of an exonuclease to liberate individual nucleotides from a strand of DNA, wherein the bases are identified in order of release, and the use of an adaptor molecule that is covalently attached to the pore in order to permit continuous base detection as the DNA molecule moves through the pore. As the nucleotide passes through the pore, it is characterized by a signature residual current and a signature dwell time within the adapter, making it possible to discriminate between non-methylated nucleotides. Additionally, different dwell times are seen between methylated nucleotides and the corresponding non-methylated nucleotides (e.g., 5-methyl-dCMP has a longer dwell time than dCMP), thus making it possible to simultaneously determine nucleotide sequence and whether sequenced nucleotides are modified. The sensitivity of nanopore sequencing can be further enhanced by optimizing salt concentrations, adjusting the applied potential, pH, and temperature, or mutating the exonuclease to vary its rate of processivity.

### C. Quantifying the Extent of Modification

In some embodiments, the present invention comprises quantifying the extent of DNA modification in at least one DNA region, wherein the extent of DNA modification in the DNA region is indicative of the accessibility of the DNA in chromatin in that region. In general, high levels of DNA modification in a DNA region, relative to a control, are indicative of a chromatin region that is in a loose or accessible configuration and that is generally transcriptionally active. Low levels of DNA modification in a DNA region, relative to a control, are indicative of a chromatin region that is in a compacted or inaccessible configuration and that is generally transcriptionally silent.

Using the nucleotide sequencing methods of the present invention, one can quantify the extent of DNA modification, e.g., methylation, by comparing the amount of modification in a DNA region to a control. In some embodiments, the amount of modification in a DNA region of a sample of interest can be quantified as a relative value by comparing to the amount of modification in a control DNA region of the sample (e.g., a DNA region that is known to be generally accessible or generally inaccessible in all cells of the sample). In some embodiments, the amount of modification in a DNA region of a sample of interest can be quantified as a relative value by comparing to the amount of modification in a corresponding DNA region of a control sample (e.g., a normal or non-diseased sample).

Quantification of modified (or unmodified) DNA regions according to the method of the invention can be further improved, in some embodiments, by determining the relative amount (e.g., a normalized value such as a ratio or percentage) of modified or unmodified copies of the DNA region compared to the total number of copies of that same region. In some embodiments, the relative amount of modified or unmodified copies of one DNA region is compared to the number of modified or unmodified copies of a second (or more) DNA regions. In some embodiments, when comparing between two or more DNA regions, the relative amount of modified or unmodified copies of each DNA region can be first normalized to the total number of copies of the DNA region. Alternatively, when obtained from the same sample, in some embodiments, one can assume that the total number of copies of each DNA region is roughly the same and therefore, when comparing between two or more DNA regions, the relative amount (e.g., the ratio or percentage) of modified or unmodified copies between each DNA region is determined without first normalizing each value to the total number of copies.

In some embodiments, the actual or relative (e.g., relative to total DNA) amount of modified or unmodified copies is compared to a control value. Control values can be conveniently used, for example, where one wants to know whether the accessibility of a particular DNA region exceeds or is under a particular value. For example, in the situation where a particular DNA region is typically accessible in normal cells, but is inaccessible in diseased cells (or vice versa), one may simply compare the actual or relative number of modified or unmodified copies to a control value (e.g., greater or less than 10% modified or unmodified, greater or less than 20% modified or unmodified, etc.). Alternatively, a control value can represent past or expected data regarding a control DNA region. In these cases, the actual or relative amount of a control DNA region are determined (optionally for a number of times) and the resulting data is used to generate a control value that can be compared with actual or relative number of modified or unmodified copies determined for a DNA region of interest.

The calculations for the methods described herein can involve computer-based calculations and tools. The tools are advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### VI. Diagnostic and Prognostic Methods

The present invention also provides methods for diagnosing or providing a prognosis for a disease or condition or determining a course of treatment for a disease or condition based on the extent and location of DNA modification in genomic DNA. In some embodiments, the DNA region is known to be differentially accessible depending on the disease or developmental state of a particular cell. In these embodiments, the methods of the present invention can be used as a diagnostic or prognostic tool. For example, in some embodiments, DNA in a target region may be highly accessible and able to be modified, e.g., by methylation, in a normal cell or tissue, whereas the DNA in that target region may be inaccessible and resistant to modification in a diseased cell or tissue (or vice versa).

Once a diagnosis or prognosis is established using the methods of the invention, a regimen of treatment can be established or an existing regimen of treatment can be altered in view of the diagnosis or prognosis. For instance, detection of a cancer cell according to the methods of the invention can lead to the administration of chemotherapeutic agents and/or radiation to an individual from whom the cancer cell was detected.

### VII. Reaction Mixtures

The present invention also provides for reaction mixtures comprising one or more of the reagents as described herein, optionally with a eukaryotic cell (whose chromatin state is to be determined). In some embodiments, the reaction mixtures comprise, e.g., a DNA modifying agent (e.g., a methyltransferase or a DNA modifying chemical) and a cell permeabilizing and/or cell disrupting agent and a eukaryotic cell. Other reagents as described herein (including but not limited to sequencing reagents) can also be included in the reaction mixture of the invention.

### VIII. Kits

The present invention also provides kits for performing the accessibility assays of the present invention. A kit can optionally include written instructions or electronic instructions (e.g., on a CD-ROM or DVD). Kits of the present invention can include, e.g., a DNA modifying agent and a cell permeabilizing and/or cell disrupting agent. DNA modifying agents can include those described herein in detail, including, e.g., a methyltransferase or a DNA modifying chemical. Kits of the invention can comprise the permeabilizing agent and the DNA modifying agent in the same vial/container (and thus in the same buffer). Alternatively, the permeabilizing agent and the DNA modifying agent can be in separate vials/containers.

The kits of the invention can also include one or more control cells and/or nucleic acids. Exemplary control nucleic acids include, e.g., those comprising a gene sequence that is either accessible in essentially all cells of an animal (e.g., a housekeeping gene sequence or promoter thereof) or inaccessible in most cells of an animal. In some embodiments, the kits include one or more sets of primers for amplifying such gene sequences (whether or not the actually gene sequences or cells are included in the kits). For example, in some embodiments, the kits include a DNA modifying agent, and a cell permeabilizing and/or cell disrupting agent, and one or more primer sets for amplifying a control DNA region, and optionally one or more primer sets for amplifying a second DNA region, e.g., a target DNA region.

In some embodiments, the kits of the invention comprise one or more of the following:
(i) a methyltransferase or other DNA modifying agent;
(ii) a cell membrane permeabilizing or disrupting agent;
(iii) a “stop” solution capable of preventing further modification by the modifying agent;
(iv) materials for the extraction and/or purification of nucleic acids (e.g., a spin column for purification of genomic DNA and/or removal of non-DNA components such as components of a “stop” solution); and
(v) reagents for the sequencing of the DNA (e.g., single-molecule real-time sequencing reagents or nanopore sequencing reagents).

### EXAMPLES

The following examples are offered to illustrate, but not to limit the claimed invention.

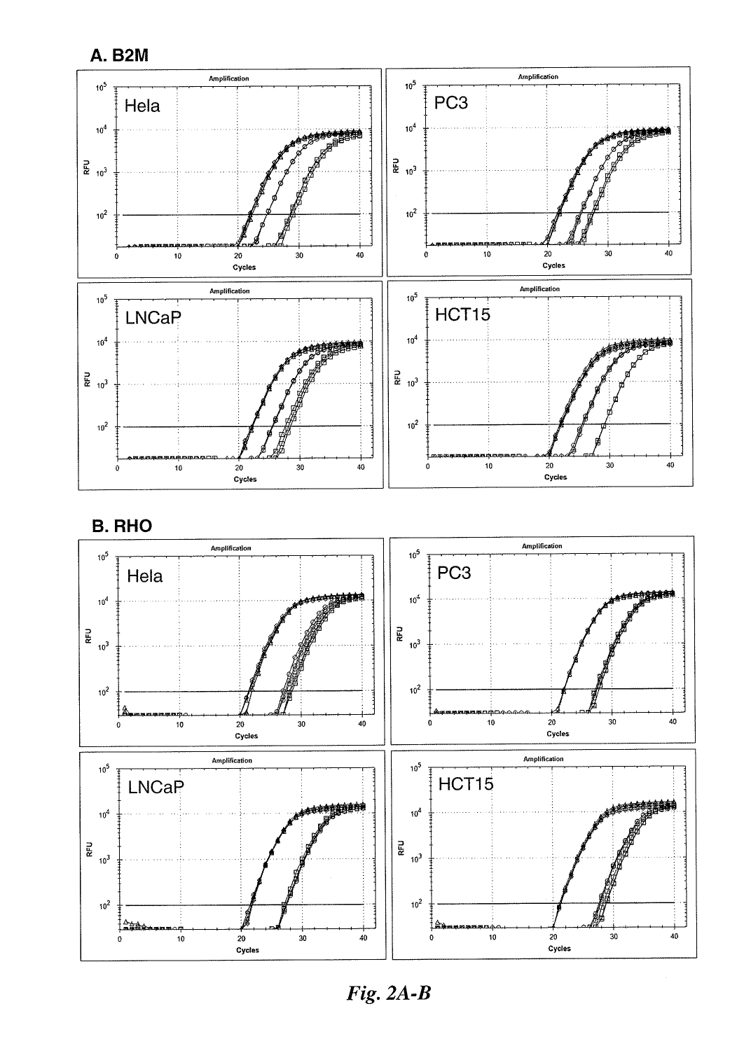

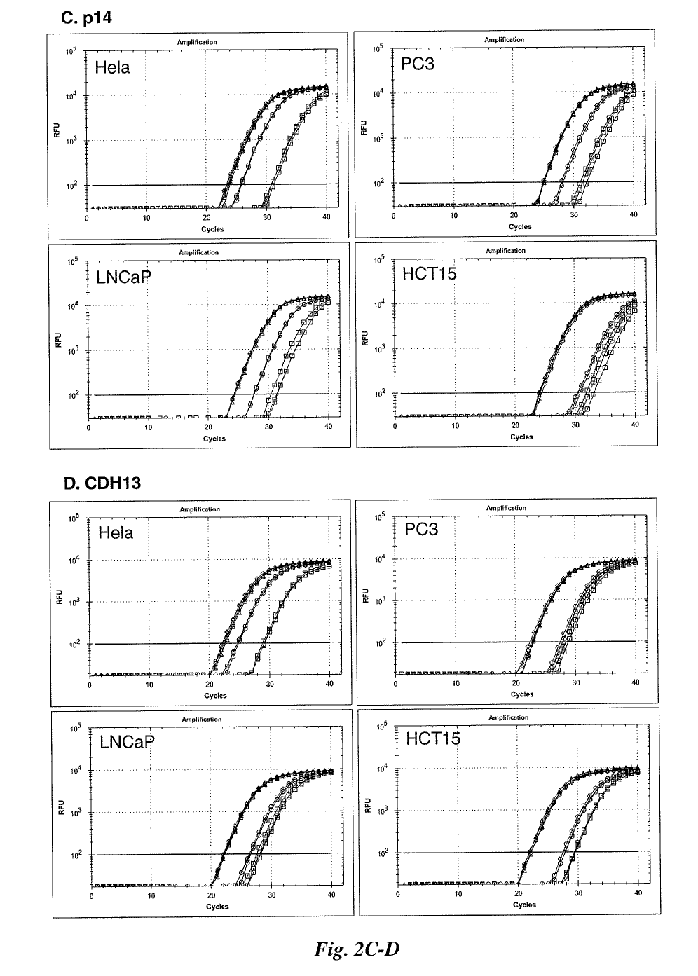

The accessibility of chromatin regions to modification by a DNA modifying agent was tested for four genes of varying levels of accessibility in four cell lines (FIG. 2). DAM methyltransferase is a bacterial enzyme that methylates adenine at the 6′ position in a GATC motif.

DNA modification in four genes—rhodopsin (RHO), beta-2 microglobulin (B2M), P14, and H-cadherin (CDH13)—was analyzed as described herein using four cell lines: HeLa, PC3, LNCaP, and HCT15. For each gene and each cell line, permeabilized cells were treated with DAM methyltransferase (no DAM treatment was used as a control). Genomic DNA was isolated, then digested with DpnII (no DpnII treatment was used as a control). DpnII digestion of selected genomic regions was assessed using quantitative PCR (qPCR) methods known in the art. For each of the amplified regions, there was one potential DAM modification site (GATC).

Following DNA modification of the genomic DNA with DAM methyltransferase, the extent of DNA modification (and therefore the level of accessibility of the genomic DNA region) was quantitated using the methylation-sensitive restriction enzyme DpnII; however, other methods such as SMRT sequencing or nanopore sequencing would also be suitable for analyzing the extent of DNA modification. DpnII is an enzyme that recognizes and digests GATC regions in unmethylated DNA, but DpnII enzymatic activity is blocked by DAM methylation; therefore, adenosine-methylated GATC motifs in DNA regions are protected from digestion.

### Analysis of the B2M Promoter

B2M is a housekeeping gene that is expressed constitutively in all cell lines. In all cell lines, the plus DAM/plus DpnII line (circle) is left-shifted from the no DAM/plus DpnII line (square) (FIG. 2A). This indicates that DAM has modified the B2M promoter and has protected it from DpnII digestion and suggests that the B2M promoter is accessible in all cell lines, a finding that is consistent with previous data.

### Analysis of the RHO Promoter

RHO is not expressed in all cell lines analyzed and its promoter is in an inaccessible chromatin configuration. In all cell lines, the plus DAM/plus DpnII line (circle) co-traces with the no DAM/plus DpnII line (square) (FIG. 2B). This indicates that the RHO promoter is protected from DpnII digestion, consistent with its location in inaccessible chromatin.

### Analysis of the p14 Promoter

p14 is not expressed in HCT15 cells and its promoter is inaccessible. p14 is expressed in Hela, PC3 and LNCaP cells and its promoter is accessible. Our analysis of DAM modification reveals that only in HCT15 cells is the p14 promoter in a predominately closed chromatin conformation (FIG. 2C).

### Analysis of the CDH13 Promoter

CDH13 is highly expressed in Hela cells and its promoter is accessible. CH13 is poorly expressed in PC3, LNCaP and HCT15 cells and its promoter is inaccessible. Our analysis of DAM modification reveals that only in Hela cells is the CDH13 promoter in a highly accessible chromatin conformation (FIG. 2D). The CDH13 promoter is moderately to tightly closed in the other cell lines.

This data demonstrates that DAM modification of chromatin in situ occurs in accessible chromatin regions but does not occur in inaccessible regions. These results also imply that by detecting modified DNA bases during DNA sequencing one can identify accessible chromatin regions.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for analyzing chromosomal DNA, the method comprising:
(a) introducing a DNA modifying agent into a nucleus having genomic DNA under conditions such that the DNA modifying agent modifies the genomic DNA in the nucleus, wherein different regions of the genomic DNA are modified to a different extent by the DNA modifying agent, thereby forming modified DNA; and
(b) nucleotide sequencing at least one DNA region in the modified DNA, wherein the sequencing comprises simultaneously determining (1) the nucleotide sequence and (2) whether sequenced nucleotides are modified.

2. The method of claim 1, wherein the nucleus is an isolated nucleus.

3. The method of claim 1, wherein the nucleus is in a cell.

4. The method of claim 3, wherein before or during step (a), the method comprises permeabilizing or disrupting a cell membrane of the cell, and wherein step (a) comprises contacting the cell with the DNA modifying agent.

5. The method of claim 3, wherein step (a) comprises expressing the DNA modifying agent in the cell, thereby introducing the DNA modifying agent into the cell.

6. The method of claim 1, wherein the modifying agent is a DNA methyltransferase.

7. The method of claim 6, wherein the DNA methyltransferase methylates adenosines in DNA.

8. The method of claim 1, wherein the sequencing comprises monitoring DNA polymerase kinetics.

9. The method of claim 1, wherein the sequencing comprises template-dependent replication of the DNA region that results in incorporation of labeled nucleotides, and wherein an arrival time and/or duration of an interval between signal generated from different incorporated nucleotides is determinative of the presence or absence of the modification and/or the identity of an incorporated nucleotide.

10. The method of claim 4, wherein the permeabilizing step comprises contacting the cell with an agent that permeabilizes the cell membrane.

11. A method of analyzing chromosomal DNA in a cell, the method comprising:
(a) introducing a DNA modifying agent into a nucleus having genomic DNA under conditions such that the DNA modifying agent modifies the genomic DNA in the nucleus, wherein different regions of the genomic DNA are modified to a different extent by the DNA modifying agent, thereby forming modified DNA;
(b) purifying the DNA thereby generating purified DNA;
(c) fragmenting the purified DNA;
(d) affinity purifying modified DNA from the purified and fragmented DNA, thereby generating a DNA sample enriched for modified DNA; and
(e) detecting a presence, absence, or quantity of one or more DNA region in the DNA sample enriched for modified DNA or cloning, isolating, or nucleotide sequencing at least one DNA fragment from the DNA sample enriched for modified DNA.

12. The method of claim 11, wherein the nucleus is an isolated nucleus.

13. The method of claim 11, wherein the nucleus is in a cell.

14. The method of claim 13, wherein before or during step (a), the method comprises permeabilizing or disrupting a cell membrane of the cell, and wherein step (a) comprises contacting the cell with the DNA modifying agent.

15. The method of claim 13, wherein step (a) comprises expressing the DNA modifying agent in the cell, thereby introducing the DNA modifying agent into the cell.

16. The method of claim 11, wherein the modifying agent is a DNA methyltransferase.

17. The method of claim 16, wherein the DNA methyltransferase methylates adenosines in DNA.

18. The method of claim 11, wherein the affinity purifying comprises contacting the fragmented and purified DNA with a protein affinity agent having affinity for modified DNA under conditions to allow for binding of the affinity agent to modified DNA, and removing DNA that does not bind to the affinity agent.

19. The method of claim 11, wherein the detecting step comprises detecting the quantity of copies of at least one DNA region in the DNA sample enriched for modified DNA.

20. The method of claim 11, wherein the method comprises amplifying the at least one DNA region.

21. The method of claim 11, wherein the detecting step comprises nucleotide sequencing at least one DNA region.

22. The method of claim 21, wherein the nucleotide sequencing comprises monitoring DNA polymerase kinetics.

23. The method of claim 21, wherein the nucleotide sequencing comprises simultaneously determining (1) the nucleotide sequence and (2) whether sequenced nucleotides are modified.

24. The method of claim 11, wherein the fragmenting comprises shearing or sonicating the DNA or digesting the DNA with a sequence non-specific nuclease.
